(19) United States
(12) Patent Application Publication
(10) Pub. No.: US 2009/0209623 A1
(43) Pub. Date: Aug. 20, 2009

(54) Anti-sense nucleic acid derived from organism

(76) Inventors: Jon Tomono, Akashi-shi Hyogo (JP); Souichi Morikawa, Himeji-shi Hyogo (JP); Takahisa Nakai, Kobe-shi Hyogo (JP)

Correspondence Address: BIRCH STEWART KOLASCH & BIRCH, PO BOX 747, FALLS CHURCH, VA, 22040-0747, US

(21) Appl. No.: 11/989,301

(22) PCT Filed: Jul. 24, 2006
(86) PCT No.: PCT/JP2006/314597

§ 371 Date: Jan. 24, 2008

(30) Foreign Application Priority Data
Jul. 25, 2005 (JP) 2005-214041
May 29, 2006 (JP) 2006-148621

(51) Int. Cl. A61K 31/7088; C07H 21/04; A61K 48/00

(52) U.S. Cl. 514/44 A; 536/24.5

(57) ABSTRACT

Disclosed are: a method for producing an anti-sense nucleic acid which has a wide variation in nucleotide sequences and excellent anti-sense properties at a low cost in a simple manner and in a large scale; and a cosmetic composition or pharmaceutical composition comprising the anti-sense nucleic acid. A method is discovered which can produce a low molecular weight nucleic acid having anti-sense properties from a nucleic acid starting material derived from an organism at a high yield by using a probe nucleic acid having at least a part of the nucleotide sequence of a target gene. An anti-sense nucleic acid produced by using a nucleic acid derived from an organism as a starting material; and a method for producing the anti-sense nucleic acid.

### TECHNICAL FIELD

The present invention relates to an anti-sense nucleic acid made from a nucleic acid derived from an organism, and a method of producing the anti-sense nucleic acid. The present invention also relates to a cosmetic composition, a skin preparation for external use, or a pharmaceutical composition, each containing the anti-sense nucleic acid derived from an organism.

### BACKGROUND ART

Following the event that the Human Genome Project has revealed the base sequences constituting the human genome DNA, it is now expected that development of effective methods for knocking out genes leads to application of familiar fields such as cures for many intractable diseases, health, and beauty. In this circumstance, effectiveness of anti-sense nucleic acids has drawn attention in the fields of biology and medical science as a material that inhibits activity of genes.

This technique is to design and artificially synthesize an anti-sense nucleic acid based on base-sequence information on a particular target gene, and place the anti-sense nucleic acid in a living organism to inhibit only the effect of the gene. This allows application to the study of the functions of a gene and utilization to inhibit production of a particular protein causing a disease. It is needless to say that this technique is utilized in the study of biochemistry and molecular biology, and the latter leads to pharmaceutical development.

Especially anti-sense nucleic acid medicines as well as genetic curing medicines are expected to be a new type of medicine of the 21^{st} century. In recent years, there has been a movement of applying the anti-sense nucleic acids to skin-lightening cosmetic products (Patent Literature 1). However, application to the medicines and cosmetic products requires chemical synthesis of a considerable amount of the anti-sense nucleic acids, which are active ingredients thereof, with the use of nucleic acid synthesizing machines, and a vast amount of cost is required to obtain the amount by which the nucleic acids are to be added to a product. Further, as the synthetic anti-sense nucleic acid thus obtained is a chemical synthetic, a little knowledge in regard to safety has been available. This has hindered the application to the humans.

Further, the anti-sense nucleic acid, first of all, needs to be hybridized with a target gene or a transcription RNA thereof to produce an effect (antisense effect) of inhibiting expression of the gene. Methods of preparing a nucleic acid having the antisense effect include: carrying out experiments using many synthesized anti-sense nucleic acids to select a more effective nucleic acid (Nonpatent Literatures 1, 2); and predicting and synthesizing, in accordance with a certain algorithm, a base sequence having the antisense effect, without carrying out an experiment (Nonpatent Literature 3).

However, it is necessary with the former method to prepare a partial sequence complementary to a target gene and to actually carry out experiments on the target gene to find out whether or not the sequence has the antisense effect. This requires time, cost, and effort. In regard to the latter method, it is the fact that no universal algorithm appropriate for every case has been found out, yet. Thus, a method of producing an anti-sense nucleic acid that allows limitation on gene expression safely, inexpensively, and more reliably has been demanded.

### Patent Literature 1: Published Japanese Translation of PCT International Patent Application Publication No. 2003-521929

### Nonpatent Literature 1: K. R. Blake et al., Biochemistry, Vol. 24, pp. 6132-6138, 1985.

### Nonpatent Literature 2: E. Uhlmann, A. Peyman, Chemical Reviews, Vol. 90, pp. 543-584, 1990.

### Nonpatent Literature 3: R. A. Stull et al., Nucleic Acids Research, Vol. 20, pp. 3501-3508, 1992.

### DISCLOSURE OF INVENTION

### Technical Problem

The present invention has as an object to provide a simple method of producing an anti-sense nucleic acid that has abundant varieties of base sequences, is producible in mass production, is inexpensive, and has an excellent antisense effect (anti-sense characteristic), and a cosmetic composition containing the anti-sense nucleic acid, a skin preparation for external use containing the anti-sense nucleic acid, and a pharmaceutical composition containing the anti-sense nucleic acid.

### Technical Solution

The inventors of the present have diligently studied to realize a simple method of producing an anti-sense nucleic acid that is producible in mass production, is inexpensive, and has an excellent anti-sense characteristic, and finally finds out a method of obtaining, from a nucleic acid derived from an organism material, a low-molecular nucleic acid that exhibits an anti-sense characteristic, by a simple operation and in good yield.

Namely, the following are the present invention.

[1] A method of producing an anti-sense nucleic acid, the method comprising processing a material of a nucleic acid derived from an organism.
[2] The method of [1], in which the organism is a microorganism, an animal, or a plant.
[3] The method of [1] or [2], in which the nucleic acid fragmented and broken into a single strand is made to adhere to the probe nucleic acid having at least a part of a base sequence of a target gene, and, after the nucleic acid adhering to the probe nucleic acid is separated from a nonadherent nucleic acid, the nucleic acid that is single-stranded and adheres to the probe nucleic acid is peeled off from the probe nucleic acid and recovered.
[4] The method of [3], in which the probe nucleic acid is fixed to a carrier at least in the step of separating the nucleic acid adhering to the probe nucleic acid.
[5] The method of [3], in which an ultrafilter membrane is used in the steps of separating and/or recovering the adherent nucleic acid adhering to the probe nucleic acid.
[6] The method of [5], including the following steps (a) to (d) of:

(a) fragmenting and fractionating the nucleic acid derived from an organism;

(b) dissociating a fragment of the nucleic acid thus fragmented and fractionated into a single strand, and making the fragment adhere to the probe nucleic acid;

(c) separating and removing the nonadherent nucleic acid by use of an ultrafilter membrane; and

(d) peeling off, from a probe nucleic acid, the fragment of the nucleic acid thus dissociated into the single strand, and recovering the fragment by use of the ultrafilter membrane.

[7] The method of [6], in which steps (b) to (d) are repeated by use of a probe nucleic acid having a molecular weight greater than an exclusion molecular weight of the ultrafilter membrane used in the steps of separating and recovering.
[8] An anti-sense nucleic acid, characterized by being derived from an organism.
[9] The anti-sense nucleic acid of [8], in which the organism is a microorganism, an animal, or a plant.
[10] An anti-sense nucleic acid, having a range in nucleic acid type.
[11] An anti-sense nucleic acid, obtained by use of the method defined in [3].
[12] The anti-sense nucleic acid defined in any one of [8] to
[11], in which the anti-sense nucleic acid is to be hybridized with a melanin synthesis pathway related gene, a wrinkle formation related gene, an age-related gene, a hair-growth related gene, or a cancer-related gene.
[13] The anti-sense nucleic acid of [12], in which the nucleic acid is to be hybridized with a human tyrosinase gene or a human MMP-1 gene.
[14] A cosmetic composition or a skin preparation for external use, each containing, as an effective ingredient, an anti-sense nucleic acid defined in any one of [8] to [13].
[15] A pharmaceutical composition, containing, as an active ingredient, an anti-sense nucleic acid defined in any one of [8] to [13].

### ADVANTAGEOUS EFFECT OF THE INVENTION

With the present invention, it is possible with a simple method to produce, by use of a nucleic acid derived from an organism, an anti-sense nucleic acid that has abundant variations of base sequences, is producible in mass production, is inexpensive, and has an excellent anti-sense characteristic. The obtained anti-sense nucleic acid derived from an organism is applicable to cosmetic compositions or pharmaceutical compositions.

### BRIEF DESCRIPTION OF DRAWINGS

FIG. 1 MMP-1 inhibition effects of an anti-sense nucleic acid of the present invention (western blotting result).

FIG. 2 HPLC chromatographic pattern of the anti-sense nucleic acid of the present invention.

### EXPLANATION OF REFERENCE

1: negative control
2: h-MMP1-antiDNA
3: h-MMP1-oligo

### BEST MODE FOR CARRYING OUT THE INVENTION

The following describes in detail the present invention.

An anti-sense nucleic acid in the present invention is a mass of nucleic acids containing single-stranded DNA or RNA having the length of approximately 5 bases to 100 bases, and has as an object to inhibit expression of a particular gene relating to beauty, disease and the like, in a translational level of a protein by carrying out selective annealing (adhering) to RNA in a transcriptional level thereof. Conventionally, artificially synthesized anti-sense nucleic acids have been used. In the present invention, a main aim is to make an object anti-sense nucleic acid from a nucleic acid derived from an organism. In the present invention, the anti-sense nucleic acid, derived from an organism, thus obtained has the length of 5 bases to 100 bases, preferably 10 bases to 50 bases.

In the present invention, how the anti-sense nucleic acid is produced is not particularly limited as long as the anti-sense nucleic acid is made from a nucleic acid derived from an organism, but it is preferable that the anti-sense nucleic acid be produced by the following steps of:

(1) fragmenting the nucleic acid derived from an organism material and, when necessary, dissociating the nucleic acid into single strands;
(2) causing the single-stranded nucleic acid fragment to adhere to the probe nucleic acid;
(3) separating the single-stranded nucleic acid fragment adhering to the probe nucleic acid; and
(4) recovering the anti-sense nucleic acid thus separated.

The following describes in detail the respective steps above. Note that the present invention is not limited to the method described below.

(1) Fragmenting Nucleic Acid Derived from an Organism Material and, when Necessary, Dissociating the Nucleic Acid into Single Strands

The anti-sense nucleic acid of the present invention is made from a nucleic acid derived from an organism. Examples of the organism include microorganisms, animals, and plants. Examples of eukaryotes of the microorganism include Mastigomycotina, Zygomycetes, Deuteromycetes (e.g. molds), Basidiomycetes (e.g. mushrooms), yeast and the like. Examples of prokaryotes include bacteria such as aerobic bacteria and anaerobic bacteria, Actinobacteria, Archaebacteria, Cyanobacteria and the like. Examples of the animals include mammals, reptiles, amphibians, birds, and fish. Examples of the plants include Myxomycophyta, Eumycophyta, Rhodophyta, Phaeophyta, Bryophyte, Pteridophyta, Gymnospermae, and Magnoliophyta.

The organism from which the nucleic acid is to be derived may be an organism with artificially recombined genes, for example. It is, however, preferable in view of safety that the organism be a natural organism, more preferably an organism that is available in a relatively large quantity and is harmless to humans as having been eaten by humans. The organism is not particularly limited, but a nucleic acid (DNA) derived from salmon milt of the fish corresponds to the organism.

Further, extranuclear DNA derived from an organism, such as plasmid DNA, phage DNA, and virus DNA, is also utilizable as the nucleic acid ingredient. The plasmid DNA is not particularly limited, and examples of the plasmid DNA include pUC-type and pBR-type, both of which are replicable in Escherichia coli. The phage DNA is not particularly limited, and examples of the phage DNA include phage lambda and φX174. Further, the nucleic acids that are used as material may be either of single-stranded or double-stranded. It is also possible by use of nucleic acid sequence information on database to select an organism species that has many sequences that are considered to exhibit object antisense effects.

In the present invention, the above-described nucleic acid derived from an organism is fragmented in such a way as to contain many groups of nucleic acids each having the length of, preferably, 2 bases to 200 bases, more preferably the length of 5 bases to 100 bases, and even more preferably the length of 10 bases to 50 bases, in view of anti-sense effects. This fragmentation may be physical fragmentation, enzymatic fragmentation, or chemical fragmentation. The physical fragmentation is not particularly limited, and examples of the physical fragmentation include fragmentation by water, the air, physical pressure such as friction, ice, or ultrasonic waves. The enzymatic fragmentation is not particularly limited as long as an enzyme that can fragment the nucleic acid is used, and examples of the enzyme include Deoxyribonucleas I (DNase I) derived from various organisms, such as Escherichia coli, and restriction enzymes. Further, the fragmentation may be carried out by a combination of the physical fragmentation, the enzymatic fragmentation, and the chemical fragmentation.

It is also possible to fractionate the nucleic acid thus fragmented, whereby nucleic acid fragments are uniformed in size. The way of the fractionation is not particularly limited, but fractionation by use of ultrafilter membranes or the like is preferable in view of operability and the processing amount. Short nucleic acids having bases fewer than five are removed by processing with an ultrafilter membrane of approximately 2000 exclusion molecular weight. Long unnecessary nucleic acids having several hundred bases or more are removed by processing with an ultrafilter membrane of approximately 15000 to 30000 exclusion molecular weight. Combination of the foregoing enables fractionation into a suitable mass of nucleic acids having a certain degree of uniformity in length, for example with the molecular weight of 2000 to 15000, 2000 to 20000, or 2000 to 30000. It should be noted that the ultrafilter membranes in the present Specification include not only common ultrafilters but also so-called dialysis membranes.

Next, if the ingredient, which is the nucleic acid derived from an organism, is double stranded, the nucleic acid thus fragmented is dissociated into single strands by use of a known method such as thermal or alkali treatments. To make the nucleic acid fragments uniform in size by use of ultrafilter membranes, it is possible to carry out this dissociating into single strands before, after, or concurrently with the fractionation by ultrafiltration. Alternatively, it is also possible to carry out the dissociation into single strands concurrently with adhesion in a process of adhering to the probe nucleic acid, which process will be described later. In this case, a mass of double-stranded nucleic acid fragments are brought into contact with the probe nucleic acids and mixed with the probe nucleic acids, and this is followed by dissociation into single strands and adhesion in this reaction system. This is also a preferred embodiment of the production method of the present invention.

### (2) Making Single-Stranded Nucleic Acid Fragment Adhere to Probe Nucleic Acid

In the present invention, it is preferable that an object anti-sense nucleic acid be separated from a mass of the nucleic acid fragments obtained, in a sequence-specific manner by use of a probe nucleic acid. The “probe nucleic acid” in the present invention is a single-stranded nucleic acid that has at least a part of the base sequences of a sense strand of a gene that is to be targeted (gene whose action is desired to be inhibited). Use of the probe nucleic acid makes it possible to separate, from the mass of nucleic acid fragments obtained in (1), the anti-sense nucleic acid for inhibition effect of a target gene, utilizing the behavior that only the object anti-sense nucleic acid anneals with (adheres to) the probe nucleic acid.

A gene (target gene) that is to be targeted by the anti-sense nucleic acid of the present invention is not particularly limited as long as the gene relates to beauty, disease or the like, and an appropriate gene is selectable according to the purpose. Concrete examples thereof include a melanin synthesis/transport related gene cluster (melanin synthesis pathway related genes), a wrinkle formation related gene cluster, an age-related gene cluster, a hair-growth/loss related gene cluster, a cancer related gene cluster, and the genes that will be described below. It is also possible to select a combination of two or more kinds from the foregoing genes as the target gene.

As the melanin synthesis pathway related gene cluster, for example genes that relate to development or differentiation of tyrosinase, trp-1, trp-2, or melanocyte may serve as the target. For example, pax3, sox10, and MITF, which are master genes in differentiation from neural crest cells into melanocyte, may serve as the target. Further, in regard to the target, the genes themselves may be targeted directly, genes that control expression of the genes may be targeted, or genes that are involved in maturity of gene products thereof, for example genes involved in addition or breakage of sugar chains, may be targeted.

For example, it is known that, in expression regulation, MITF-M binds to upstream M-box to enhance expression of a tyrosinase gene, a trp-1 gene, and a trp-2 gene. It is predicted that use of a specific anti-sense nucleic acid in MITF-M sequence allows those three genes to be controlled. It is also possible to inhibit an expression system of a regulator controlling expression of individual genes. For example, it is known that metallothionein is involved in regulation of expression of tyrosinase (retaining the metallothionein is beneficial), Effects are obtainable by controlling expression of a protein that is involved in this inhibition of expression or in degradation.

Further, it is also possible to block a signal transducing pathway that is involved in melanin production. For example, it is known that binding of signals α-MSH to MC1R receptors on a surface of melanocyte enhances eumelanin synthesis. It is also known that ET-1 signals bind to ETRA receptors and SCF signals bind to c-kit receptors. It is considered that, for example, restricting systems of expression of α-MSH and ET-1 by use of the anti-sense nucleic acid is also possible. Further, a gene cluster that is involved in melanin transport is also a potential target. Melanin is produced in melanocyte, transported to keratinocyte, and distributed to skin. For example inhibiting PAR2 involved in this transport is expected to repress deposition of melanin on a surface of the skin.

Examples of the wrinkle formation related gene cluster include genes coding for proteins degrading collagen, hyaluronic acid, or elastin, all of which constitute dermis that plays role in supporting skin and keeping skin tone and firmness. Concretely, genes of collagenase such as matrix metalloproteinase (MMP), genes of hyaluronidase, and genes of elastase are exemplified. Especially collagen constituting collagenous fibril occupies approximately 90% of dermis and functions as a backbone supporting skin. As being known to decrease with advancing age, the collagen is considered as a promising target of anti-wrinkle.

An example of the age-related gene cluster is a telomerase-regulating gene cluster. A series of nucleic acids extending from a periphery of chromosome is called telomere. The telomere plays a role in protecting chromosome, and at the same time, becomes shortened each time a cell repeats division. Thus, the telomere is considered to determine the lifespan of cells. Telomerase, which is regeneration enzyme, repairs this shortening telomere. It is predicted that if activity of the telomerase is maintained at a normal level, cellular aging is radically prevented. In other words, it is expectable that expression of the telomerase-regulating gene cluster controlling telomerase activity is inhibited with the anti-sense nucleic acid to control aging. Further, it is known that this telomerase activity is enhanced extraordinarily in a cancer nest. It is predicted that inhibiting telomerase enzyme activity with the use of the anti-sense nucleic acid of the present invention helps cancer care.

Examples of the hair-growth/loss related gene cluster include those coding for hair-shaft regulating proteins and/or structural proteins. Concrete examples of the regulating proteins and structural proteins of hair shafts include: proteins involved in cell cycle; proteins relating to anti-growth, such as IGF receptors and T4 thyroid receptors; proteins relating to anti-damage, such as cytokines, including IL1, IL6, TNFα, and MCP1, MMP, urokinase, and lipoxygenase; proteins relating to regrowth, such as proteins involved in degradation of PGF2α, 5αreductase; related proteins focusing on morphosis, such as α3β1 integrin, beta-catenin, laminin 10, and LEF-1; proteins involved in formation of hair shafts (e.g. curling or uncurling) and differentiation of hair shafts (e.g. acidic or basic cornified hair); and proteins relating to formation of hair shafts, such as enzyme (e.g. thiol oxidoreductase, transglutaminase 3, transglutaminase 5) relating to cross-linking of hair shaft proteins.

Thereamong, 5α reductase is known to reduce testosterone, and the resulting product is known to repress cell division of hair matrix cells. Inhibiting expression of genes of the enzymes with the use of the anti-sense nucleic acid makes it possible to aim at hair-growth effect. Further, inhibiting expression of a particular gene with the use of the anti-sense nucleic acid makes it possible to enhance activity of cells relating to hair formation, such as hair follicle cells, hair matrix cells, and hair germ cells, and to adjust their cell cycles. For example, the use of the anti-sense nucleic acid is applicable to obtain effects of shortening the telogen and preventing cell suicide (apotosis) (inhibiting activity of suicidal enzyme caspase). Further, regarding hair loss, there is a possibility that the hair loss is restrained by inactivating a receptor gene of Bone Morphogenetic Protein (BMP) involved in cell proliferation and differentiation control.

The cancer-related gene cluster is not particularly limited. Examples of the cancer-related gene cluster include, genes called oncogenes, such as ras, and genes that are considered to be involved in cancerous activation, such as c-fos, c-myc. Further, an anti-sense nucleic acid targeting on a gene that is involved in viral infection, replication of virus, and the like is also usable as an anti-virus agent.

Further, genes involved in cytodifferentiation or growth phenomenon are considered to be a target of the anti-sense nucleic acid of the present invention. Concretely, examples of the genes include the following genes that codes for protein:

growth factors, such as EGF, TNF-α, TGF, endothelin, NGF, HGF, IGF, and VEGF;

IL1, IL6, or IL8 type cytokines;

EGFr, TGFr, PAR, PPAR, FXR, RXR, CB1R, CB2R, VR1, CRAB2, or u-PA type receptors;

calmodulin, CLP or CLSP type calcium-binding proteins;

calcium-binding protein of S100 protein family, such as S100A8, S100A9, and S100A7;

transglutaminase, such as transglutaminase 1, 3, or 5;

proteins that ensure intercellular cohesion/binding, such as occludin, laminin, caveolin, desmoglein, desmocollin, corneodesmosin, plakoglobin, and desmoplakin;

enzymes involved in protein posttranslational modification, such as phosphatase and proteinphosphatase;

calcineurin, phosphorylase, protein kinase (e.g. PKC), glucosyltransferase, peptidylarginine deiminase;

proteases, such as MMP, 1-, 2-, 3-, or 9-elastase, aspartic protease (e.g. cathepsin E and cathepsin D), cathepsin L, B or H-type cysteine protease, cathepsin L2, SCCL, chymotrypsin equivalent, SCCE (kallikrein 7) type trypsin-like protease, SCTE (kallikrein 5) type urokinase, SASPase, caspase (especially caspase 14), calpain, subtilisin-like proprotein convertase-type protease involved in hydrolytic degratation of filaggrin (e.g. Furin, PACE4, PC5/6 and PC7/8), and protease of intermembrane-type serine protease family (e.g. matriptase);

exoglycosidase or endoglycosidase, such as heparanase type hyaluronidase, chondroitinase, aspartylglucosaminidase, B glycosidase, and glycosidase;

lipid-metabolizing enzyme, such as HMG-CoAreductase, cholesterol sulfatase, sulfotransferase, sphingomyelinase, and ceramidase;

eicosanoid metabolizing enzyme, such as cyclooxygenase, lipoxygenase, phospholipase, and 15-PGDH;

hormone-metabolizing enzymes, such as type I or type II 5α reductase;

matrix proteins of elastin-type, collagen-type and the like;

proteins involved in skin hydration cytokeratin type keratinocyte differentiation protein, filaggrin, and water channel;

proteins involved in antibacterial skin protection, such as hBD2, hBD3, Dermcidin, and ribonuclease 7; and

proteins involved in maturation of dermis, such as lysyl oxidase and lysyl hydroxylase.

Other than those mentioned above, targeting a serine protease gene such as urokinase and repressing it with the anti-sense nucleic acid of the present invention are considered effective for beauty cures and improvement in appearance of skin dehydrated or inflamed.

Further, genes of the following proteins may be targeted in the anti-sense nucleic acid of the present invention, for example: proteins involved in synthesis of sebum as application to skin type improvement portion (e.g. HMG-CoAreductase and squalene synthase); bacterial lipase as application for the purpose of inhibiting formation of dandruff; lox12 and/or cox2 and/or IL1 and/or TGFβ1 as application for the purpose of inhibiting loss of hair; abnormal proteins and/or oligopeptide for the purpose of inhibiting expression (especially those related to virus activity or cancer cells such as HPV, or those overexpressed in certain types of diseases); proteins relating to growth of certain types of cytokines (e.g. IL1, TNFα-308 and TNFβ+252), receptor proteins (e.g. Toll-like receptor, TLR), phosphatidylinositol 3 kinases, cell adhesion molecules (e.g. CDw60) and the like; protease, especially serine protease (cornified layer chymotrypsin enzyme, SCCE) or metalloprotease (especially, MMP-9 and MMP-19 involved in psora); and calcium binding protein (calmodulin-like serine protease, CLSP).

In the present invention, a target gene relating to a target whose activity is desired to be inhibited is determined from the foregoing gene clusters according to the purpose of use. On the basis of base-sequence information thereof, a probe nucleic acid having a part of or all of the base sequences of the target gene is produced. If a part of the sequences of the target gene is to be used, normally a sense strand sequence situated upstream or in the vicinity of an initiation site of translation of the target gene. The probe nucleic acid is artificially produced with the use of DNA synthesizing machines or the like. Further, it is also possible to use a gene that is single-stranded by separating the target gene from the living organism and, when necessary, fragmenting a necessary portion and carrying out amplification.

In the case in which a part of the base sequences of the target gene is to be used, the lengths of the base sequences are not particularly limited, but 2 bases to 300 bases are preferable, 5 bases to 200 bases are more preferable, and 10 bases to 100 bases are even more preferable. Regarding a preparation method to be employed in synthesizing the probe nucleic acid, for example the phosphoroamidite method (see the Procedure Manual of ABI or F. Eckstein, Oligonucleotides and Analogues: A Practical Approach, IRL Press, 1991) may be used with a DNA synthesizing machines manufactured by ABI (Applied Biosystems Inc.), DNA/RNA synthesizing machines manufactured by ABI, or automatic nucleic-acid synthesizing machines manufactured by PerSeptive Ltd. It is also possible to synthesize a nucleic acid having a desired length by PCR (polymerase chain reaction) with the use of the synthetic DNA as a primer. Further, it is also possible to use the probe nucleic acid having undergone processing for preventing deterioration due to single-stranded DNA degradation enzymes. For example, it is also possible to use a probe nucleic acid with its phosphate-bonded site being thioesterified.

An obtained crude product of the probe nucleic acid is purified by use of a common purification method, for example various chromatographies including reversed phase chromatography, ion-exchange chromatography, and high-performance liquid chromatography (HPLC) based on the principles of gel filtration chromatography, supercritical chromatography, and the like, an ethanol precipitation methods, and electrophoresis methods. Other than those mentioned above, a cartridge produced on the basis of the principles of the reversed phase chromatography (e.g. Sep-Pak Plus using tC18 as filler (long body/ENV); manufactured by PerSeptive Ltd.) or the like is employed in this purification. Note that the degree of purity thereof is confirmable by analysis using HPLC or capillary electrophoresis.

Next, the probe nucleic acid is annealed with (is caused to adhere to) the object single-stranded nucleic acid (anti-sense nucleic acid) included in the mass of the nucleic acid fragments obtained in (1). The method of annealing the probe nucleic acid and the single-stranded nucleic acid fragment is not particularly limited, and publicly known physical methods or chemical methods may be employed.

A common physical method is, for example, mixing the probe nucleic acid and the single-stranded nucleic acid fragment together and annealing under the condition of temperature close to or not higher than the value of Tm of the probe nucleic acid. Further, adjusting the temperature of this annealing enables adjustment of the degree of sequence specificity of the anti-sense nucleic acid that is to be obtained. For example, in the case of annealing at a higher temperature, that is a temperature close to the value of Tm of the probe nucleic acid, an anti-sense nucleic acid having a base sequence that is more pursuant to the base sequence of the probe nucleic acid and is higher in homology is easier to anneal. On the other hand, in the case of annealing at a lower temperature, that is, a temperature lower than the value of Tm of the probe nucleic acid, an anti-sense nucleic acid having a base sequence that shares lower homology with the base sequence of the probe nucleic acid is also easy to anneal. In this case, there is a possibility that the anti-sense nucleic acid obtained is low in antisense effect, but more fragments are annealed so that the yield becomes obtainable. Further, changing an annealing temperature stepwise in the same reaction system allows specificity and yield to become easily obtainable.

Further, as described above, it is preferable that a mass of double-stranded nucleic acid fragments be prepared in (1), and dissociation into single strands and annealing with the probe nucleic acid be carried out sequentially in a reaction system of the present step. Concretely, processing by adding heat (e.g. 90° C. or higher, preferably 94° C. or higher, more preferably 100° C. or higher) is carried out to dissociate a double strand into single strands, and then annealing is carried out by causing a reaction with the probe nucleic acid at a temperature equal to or below the value of Tm of the probe nucleic acid. In this case, mixing of the nucleic acid fragment and the probe nucleic acid together may be carried out either before or after dissociating the nucleic acid fragment into single strands.

Further, as a chemical method, in the case in which the double-stranded nucleic acid fragments are processed with an alkali such as approximately 0.01 N to 1 N sodium hydrate solution to make them into single strands in (1), it is possible to anneal the probe nucleic acid and an object anti-sense nucleic acid by making a neutrality or acidic condition after the mixing with the probe nucleic acid.

Further, a nucleic acid fragment that is not annealed with a probe nucleic acid can be reused at separation in the other probe nucleic acids.

### (3) Separating Single-Stranded Nucleic Acid Fragment Adhering to Probe Nucleic Acid

Next, the probe nucleic acid and the object anti-sense nucleic acid adhering thereto are separated from the mass of nucleic acids after the annealing reaction. There are two methods in the present invention—(A) a carrier immobilization method and (B) an ultrafilter membrane method. The following describes in detail the respective methods.

### (3-A) Separating Single-Stranded Nucleic Acid Fragment by Carrier Immobilization Method

In the carrier immobilization method, before or after the process (2) of causing the single-stranded nucleic acid fragment and the probe nucleic acid to adhere, the probe nucleic acid is fixed to a carrier that can fix the probe nucleic acid. The way to fix the probe nucleic acid to the carrier is not particularly limited. A possible way is that a special label is set in a 5′ or 3′ terminal in advance during the stage of synthesis of the probe nucleic acid to allow the probe nucleic acid to adhere to the carrier via the label. An exemplary method is to label the 5′ terminal of the probe nucleic acid with biotin and then bind the 5′ terminal to streptavidin on the carrier. It is also possible to epoxidize, carboxidize, or cyanogens brominate the carrier to allow the probe nucleic acid to bind. Alternatively, it is also possible to synthesize the probe nucleic acid directly onto a specific carrier during the stage of synthesis.

The carriers employed herein are not particularly limited, as long as the carriers are those commonly used and can fix the nucleic acids. Examples of the carriers include magnetic carriers having magnetism, glass carriers, Toyopearl (manufactured by Tosoh Corporation), and chemical synthetic carriers such as silica gel. Use of a magnetic carrier or a carrier having anion absorptive property is preferable.

In the case in which the magnetic carrier is employed as the carrier, the probe nucleic acid fixed to the magnetic carrier and the single-stranded nucleic acid fragment adhering thereto are easily separated from a nonadherent nucleic acid fragment by utilizing the magnetic force. In the case in which a carrier other than the magnetic carrier is employed as the carrier, it is possible by use of, for example, filtration or a column to separate the nonadherent nucleic acid fragment from the carrier, the probe nucleic acid fixed to the carrier, and the single-stranded nucleic acid fragment adhering to the probe nucleic. In the foregoing processes of separation, it is possible to completely remove the nonadherent nucleic acid fragments by repeating cleaning operation upon necessity.

### (3-B) Separating Single-Stranded Nucleic Acid Fragment by Ultrafilter Membrane Method

In the ultrafilter membrane method, the property of the ultrafilter membrane that allows separation by difference in molecular weight is utilized to separate the nonadherent single-stranded nucleic acid fragment from the probe nucleic acid and the object single-stranded nucleic acid adhering thereto. The following describes an exemplary detailed method thereof.

First of all, the mass of nucleic acid fragments is obtained in (1). At this time, the ultrafilter membrane is used as described above to make the molecular weight, especially the upper limit of thereof, of the nucleic acid fragment uniform. For example, an ultrafilter membrane having the exclusion molecular weight of X is employed to prepare a mass of nucleic acids that pass through the ultrafilter membrane, that is to say, a mass of nucleic acids having the molecular weight with the upper limit X. This is followed by preparation of the probe nucleic acid. At this time, either the length of the base sequence in the target gene, which base sequence is to be fragmented is adjusted, or a specific base sequence is repeated, in order to make the molecular weight of the probe nucleic acid greater than X. Annealing the probe nucleic acid with the mass of nucleic acid fragments always results in the probe nucleic acid and the single-stranded nucleic acid fragment adherent thereto having the molecular weight equal to or greater than X. Thus, use of the ultrafilter membrane having the exclusion molecular weight of X makes it possible to separate nonadherent nucleic acid fragment having the molecular weight equal to or smaller than X. The exclusion molecular weight X of the ultrafilter membrane is selectable according to a desired length of the anti-sense nucleic acid. Preferably, the exclusion molecular weight X is settable in the range of 15000 to 30000.

Needless to say, the ultrafilter membrane method is not limited to what described above, and the ultrafilter membrane method may be a method in which the difference of the molecular weight between the nonadherent nucleic acid fragment and the probe nucleic acid or the nucleic acid fragment adherent thereto is applied.

The carrier immobilization method and the ultrafilter membrane method are both selectable according to the purpose. For example, the carrier immobilization method requires a fewer number of processes and a shorter operation hours (it is not necessary to unify the nucleic acid fragments in molecular weight, and separating operation takes approximately five minutes by use of the magnetic carrier), but the ultrafilter membrane method is superior in view of the recovery rate and industrially inexpensive production.

### (4) Recovery of Anti-Sense Nucleic Acid Separated

A publicly known physical method or chemical method is employable to peel off and recover, from the probe nucleic acid, the anti-sense nucleic acid separated from the nonadherent nucleic acid. An exemplary physical method is a thermal treatment, and an exemplary chemical method is use of a sodium hydrate solution to change pH. The physical method and the chemical method are not limited to the foregoing methods.

In the case of the carrier immobilization method, the single-stranded nucleic acid peeled off (dissociated) from the probe nucleic acid is recoverable in the same manner as the separation method of the carrier and the nonadherent nucleic acid fragment. For example in the case in which the magnetic carrier is employed, the magnetic carrier and the probe nucleic acid fixed to the magnetic carrier are separable and removable by use of the magnetic force so that it becomes possible to recover only the object anti-sense nucleic acid. In the case of a non-magnetic carrier, use of filtration or a column allows the separation. In the case in which the column is used, it is possible to recover only the object anti-sense nucleic acid by, for example, cleaning and removing the nonadherent nucleic acid fragment during the separation in (3) and then carrying out the physical or chemical treatment in the column to peel off the anti-sense nucleic acid from the probe nucleic acid fixed to the carrier and to discharge the anti-sense nucleic acid from the column.

On the other hand, in the case of the ultrafilter membrane method, the physical or chemical treatment is carried out on the probe nucleic acid and the single-stranded nucleic acid fragment adherent to the probe nucleic acid, and then the object anti-sense nucleic acid dissociated is recoverable by a publicly-known method. For example, it is possible to use the ultrafilter membrane also in the recovery. Preferably, in the case in which the probe nucleic acid having the molecular weight greater than X is used in (3), use of an ultrafilter membrane having the exclusion molecular weight of X makes it possible to separate the probe nucleic acid and the object anti-sense nucleic acid and recover the object anti-sense nucleic acid. More preferably, after the nonadherent nucleic acid is cleaned in (3), the probe nucleic acid and the single-stranded nucleic acid fragment adherent to the probe nucleic acid, without modification, undergo the physical treatment or chemical treatment in the device of the ultrafilter membrane so that the anti-sense nucleic acid is peeled off from the probe nucleic acid. The anti-sense nucleic acid thus dissociated is discharged externally from the ultrafilter membrane and is recoverable directly.

The probe nucleic acid after the anti-sense nucleic acid has been peeled off and recovered is usable repeatedly in (2) to (4). Repeat use of a small amount of probe nucleic acid that is obtained by chemical synthesis and is expensive makes it possible to process a vast amount of nucleic acid derived from an organism, whereby a large amount of anti-sense nucleic acid becomes obtainable inexpensively.

The anti-sense nucleic acid thus obtained has the property of hybridizing not only with RNA in a transcriptional level in the target gene having the base sequence used in the probe nucleic acid, but also with the gene itself. Conditions of hybridization in this case are not particularly limited. Optimum conditions of hybridization of the anti-sense nucleic acid with the target gene vary according to selection of the base sequence used in the probe nucleic acid, conditions in the adhesion process, and the like. Needless to say, the anti-sense nucleic acid that is to be hybridized with the target gene even under stringent conditions is superior in anti-sense ability.

Differing from an anti-sense nucleic acid obtained by chemical synthesis, the anti-sense nucleic acid of the present invention that is made from a nucleic acid derived from an organism is constituted of nucleic acid fragments having various base sequences. Analysis of the anti-sense nucleic acid of the present invention under the conditions that allow fractionation by properties such as ionic strength or the molecular weight of the anti-sense nucleic acid by use of an ion-exchange column, a gel filtration column or the like shows that there is a pattern of bands indicating the presence of plural nucleic acids having serial sizes. This tells that the nucleic acid has a range in nucleic acid type.

The following are examples of the ion-exchange column. Exemplary anion-exchange columns include diethylaminoethyl (DEAE), quaternary aminoethyl (QAE), and quaternary ammonium (Q). Exemplary cation-exchange columns include carboxymethyl (CM), sulfopropyl (SP), and methyl sulfonate (S). Exemplary gel filtration columns include those with the use of carriers, such as Sephadex, Sephacryl, and Superose. Further, the anti-sense nucleic acid of the present invention more or less produces a desired inhibition effect in every fraction. Use of the column as described above also makes it possible to improve purity by fractionating more effective anti-sense nucleic acid fractions.

The anti-sense nucleic acid of the present invention thus prepared is usable as a gene inhibitor without modification. The anti-sense nucleic acid may be dissolved or suspended in polar solvent such as water, ethanol, or the like, or dispersed in a vehicle such as emulsion, cream, gel, ointment, or the like, and then used as the gene inhibitor.

The gene inhibitor may contain bioactive ingredients, such as active oxygen quencher, anti-inflammation agent, anti-cancer drug, skin-lightening agent, skin-cell activation agent, germicide, and the like. Other than those listed above, common medicine and cosmetic material, such as oils, surfactant, moisturizing agent, ultraviolet rays absorbing agent, powder, perfume, preservatives, and the like may be contained in the gene inhibitor.

It should be noted that, since the anti-sense nucleic acid of the present invention is constituted of nucleic acid, it is expected that the anti-sense nucleic acid alone exhibits the effects of absorbing ultraviolet rays and moistening action, which effects are those the nucleic acid originally has.

Further, in the present invention, the anti-sense nucleic acid thus obtained may be contained in a vehicle for external use to form a skin preparation for external use or a cosmetic composition. It is possible to provide the skin preparation for external use and the cosmetic composition in the form of dosage forms such as lotion, emulsion, gel, cream agent, ointment, spray agent, and the like. The skin preparation for external use and the cosmetic compositions are also providable as: skin cosmetics, such as toner, emulsion milk, cream, masks and the like; makeup cosmetics, such as makeup base lotion, makeup base cream, milky emulsion foundations, creamy foundations, ointment-type foundations, eye colors, cheek colors; body cosmetics, such as hand cream, leg cream, body lotion and the like; and other skin preparations for external use. Further, common medicine and cosmetic material, such as oils, surfactant, moisturizing agent, ultraviolet rays absorbing agent, pigments, perfume, preservatives and the like, and bioactive ingredients such as active oxygen quencher, anti-inflammation agent, skin-lightening agent, wrinkle-remedy agent, skin-cell activation agent and the like may be contained.

The anti-sense nucleic acid of the present invention is usable as a pharmaceutical composition containing the anti-sense nucleic acid as its major constituent. In this case, drug formulation is not particularly limited, as long as it is a drug formulation in which cardinal remedy is taken in either cells of an affected area or cells of an object tissue. The anti-sense nucleic acid is administered either alone or in combination with a commonly-used carrier, by way of external use, such as skin preparation for external use, other non-oral medication, or oral medication.

An anti-sense nucleic acid made from a natural nucleic acid source, especially those having been eaten by humans, by use of the method of the present invention is high in safety in the case of oral ingestion and thus preferable. When orally ingested, the anti-sense nucleic acid is considered to act in the oral cavity, throat, stomach, intestines (e.g. small intestine, large intestine), epithelial mucosa of anus, or lower tissues thereof.

The foregoing pharmaceutical composition may be administered in the form of liquid, such as solution, suspension liquid, syrup, liposome drug formulation, emulsion and the like, or in the form of solid, such as tablet, granule, powdered medicine, capsule formulation and the like. On an as-needed basis, various carriers, auxiliary agent, stabilizing agent, lubricant agent, other commonly-used additives, such as lactin, citric acid, tartaric acid, stearic acid, magnesium stearate, white kaolin, saccharose, cornstarch, talc, gelatin, agar, pectine, peanut oil, olive oil, cacao butter, ethylene glycol and the like may be added to the drug formulation.

In the case in which the anti-sense nucleic acid of the present invention is used, using, as method a preferred drug formulation, of a formulation employed in a common gene transfer is favorable, for example a membrane-fusing liposome drug formulation using Sendai virus or the like, a liposome drug formulation such as a liposome drug formulation using endocytosis or the like, a drug formulation containing a cationic lipid such as Lipofectamine (manufactured by Life Technologies Oriental INC.) or the like, a drug formulation of a combination of calcium phosphate and polyethylene glycol, or a virus drug formulation such as retrovirus vector, adenovirus vector and the like. Especially, the anti-sense nucleic acid is usable in the form of a membrane-fusing liposome drug formulation.

Further, drug formulation techniques that are used in common drug delivery systems (DDS) are applicable to the anti-sense nucleic acid of the present invention. In this case, for example PLA (polymer of lactic acid), PLGA (copolymer of lactic acid and glycolic acid) or the like is usable as a vehicle of the drug formulation. Techniques of targeting and division using this DDS technique are also usable.

In the case of the liposome drug formulation, the structure of the liposome may be any of a large unilamellar vesicle (LUV) liposome, a multilamellar vesicle (MLV) liposome, and a small unilamellar vesicle (SUV) liposome. Regarding their sizes, LUV can take a particle system of approximately 200 nm to 1000 nm, MLV can take a particle system of approximately 400 nm to 3500 nm, and SUV can take a particle system of approximately 20 nm to 50 nm. In the case of the membrane-fusing liposome drug formulation using Sendai virus or the like, use of MLV having the particle system of 200 nm to 1000 nm is preferable.

The method of producing the liposome is not particularly limited, as long as the anti-sense nucleic acid of the present invention is maintained. Commonly-used methods, such as a reversed phase evaporation method (Szoka, F. et al.: Biochim. Biophys. Acta, Vol. 601, 559 (1980)), an ether implantation method (Deamer, D. W.: Ann. N.Y. Acad. Sci., Vol. 308, 250 (1978)), a surfactant method (Brunner, J. et al.: Biochim. Biophys. Acta, Vol. 455, 322 (1976)), or the like may be employed to produce the liposome.

Phospholipid, cholesterols, nitrogen lipids and the like are used as the liquids forming the liposome structure. Generally, phospholipid is suitable. The following are also usable: natural phospholipids, such as phosphatidyl choline, phosphatidyl serine, phosphatidylglycerol, phosphatidylinositol, phosphatidyl ethanolamine, phosphatidic acid, cardiolipin, sphingomyelin, egg-yolk lecithin, soybean lecithin, lysolecithin and the like, or any of the above hydrogenated by use of a common method; and synthetic phospholipids and the like, such as dicetylphosphate, distearoylphosphatidyl choline, dipalmitoylphosphatidyl choline, dipalmitoylphosphatidyl ethanolamine, dipalmitoylphosphatidyl serine, eleostearoylphosphatidyl choline, eleostearoylphosphatidyl ethanolamine, eleostearoylphosphatidyl serine, and the like.

Lipids containing the foregoing phospholipid are usable alone or in combination of two or more types. In this case, use of those containing, in a molecule, an atom group having a cationic group such as ethanolamine, choline and the like makes it possible to increase a binding rate of electrically negative anti-sense nucleic acid. Other than those major phospholipid in liposome formation, commonly-known additives for liposome formation, such as cholesterols, stearylamine, α-tocopherol and the like are also usable.

To the liposome thus obtained, membrane-fusing promoting substance, such as Sendai virus, inactivated Sendai virus, membrane-fusing promoting protein purified from Sendai virus, polyethylene glycol and the like may be added in order to promote taking the liposome in cells of the affected area or cells of an object tissue.

To concretely describe a method of producing the liposome drug formulation, for example the liposome formative substance and cholesterol or the like are dissolved into an organic solvent, such as tetrahydrofuran, chloroform, ethanol or the like. Then, this is put into a suitable container, and solvent is distilled away under reduced pressure, whereby a membrane of liposome formative substance is formed on an inner surface of the container. Buffer containing the anti-sense nucleic acid is added and stirred. The membrane-fusing promoting substance, as desired, is added to the liposome thus obtained, and then the liposome is isolated. The liposome containing the anti-sense nucleic acid thus obtained is either suspended in a suitable solvent, or lyophilized first and then re-dispersed in a suitable solvent to be utilized for cures. It is possible to add the membrane-fusing promoting substance sometimes after the isolation of the liposome and before the use.

Further, it is possible to use the anti-sense nucleic acid of the present invention as a material for the studies of microorganism, plants, animals and the like, in the same manner as common chemical synthetic anti-sense nucleic acids.

The foregoing description discusses mainly the case of the anti-sense DNA as the anti-sense nucleic acid, but anti-sense RNA is also producible in the same manner if RNA derived from an organism is used as a constituent of the nucleic acid.

Further, application of the method of producing the anti-sense nucleic acid of the present invention makes it possible to produce not only a single-stranded anti-sense nucleic acid but also decoy nucleic acid, which is a double-stranded DNA, and siRNA, which is a double-stranded RNA. The decoy nucleic acid is a nucleic acid that aims to prevent a transcription factor from binding to a target gene and aims at inhibition in a transcriptional level. In the manner as described above, a single-stranded anti-sense DNA (anti-sense DNA: anti-sense nucleic acid of the present invention) is made from a nucleic acid derived from an organism. Further, a single-stranded sense DNA (sense DNA) is made by an applied method of that of the present invention (nucleic acid having the anti-sense base sequence of the target gene is used as the probe nucleic acid). Then, the anti-sense DNA is annealed with (caused to adhere to) the sense DNA so that the decoy nucleic acid is produced.

### EXAMPLE

The following Examples more concretely describe the present invention. It should be noted, however, that the present invention is not limited to the following Examples.

### Example 1

### Synthesis of Probe Nucleic Acid (for Evaluation of Mouse Melanoma)

All of the synthetic nucleic acids used in the present Example are nucleic acids synthesized by Sigma Genosys.

To prepare respective anti-sense nucleic acids of a tyrosinase gene and a trp-1 gene, both of which were genes constituting a mouse melanin synthetic pathway, the following probe nucleic acids were synthesized. Sequence number 1 shows 48 bases, from 51 to 4 bases upstream of a translation initiation site, of a sense sequence of a sequence of mRNA of a tyrosinase derived from a mouse (Genbank registration number: NM#011661). A probe nucleic acid obtained by adding biotin to the 5′ terminal of Sequence number 1 and then carrying out synthesis was named as bio-m-Tyr. Sequence number 2 shows 48 bases, from 50 to 3 bases upstream, of a sense sequence of a sequence of mRNA of Trp-1 derived from a mouse (Genbank registration number: NM#031202). A probe nucleic acid obtained by adding biotin the 5′ terminal of Sequence number 2 and then carrying out synthesis was named as bio-m-Trp. Further, as a negative control, a probe nucleic acid obtained by biotinylating the 5′ terminal of a base sequence of a nucleic acid (sequence number 3), which base sequence was a randomly-rearranged base sequence of sequence number 1, with the equal amounts of A, T, G, C, contained, was synthesized and named as Sh-bio-m-Tyr. Further, as a control, a synthetic single-stranded nucleic acid (chemical synthetic anti-sense nucleic acid) that was predicted to exhibit anti-sense action with respect to a mouse tyrosinase gene and a trp-1 gene was prepared and named as m-Tyr-oligo (sequence number 4) and m-Trp-oligo (sequence number 5). Note that these are lengths of 20 bases of the genes, from 47 to 28 bases upstream of the translation initiation site and 38 to 19 bases upstream of the translation initiation site, respectively.

### Example 2

### Obtaining Anti-Sense Nucleic Acid

(1) Cutting DNA Derived from Salmon Milt

To a 2.8 ml solution of 5% (w/v) DNA-Na derived from salmon milt (manufactured by Nichiro), 0.4 ml of 0.2 mg/ml DnaseI (manufactured by Amersham Bioscience), 0.4 ml of 100 mM MnCl₂, and 0.4 ml of 10× DNaseI buffer (100 mM Tris-hydrochloric acid pH7.5, 1 mM of CaCl₂) were added and then left for 10 minutes at 37° C. Thereafter, approximately 4 ml of phenol-chloroform-isoamyl alcohol solution (manufactured by Nacalai) was added and centrifuged for 10 minutes at 17,800×g, and approximately 4 ml of supernatant was recovered. Then, 8 ml of 100% ethanol and 2 ml of 2.5M ammonium acetate, both of which had been cooled in advance, were added to the supernatant and left for approximately two hours at −20° C. This was centrifuged for 10 minutes at 17,800×g, and 70% ethanol with respect to a precipitate thus obtained was added. This was centrifuged for another five minutes at 17,800×g. The supernatant was discarded, and the precipitate was dried with a centrifugal evaporator. The precipitate was dissolved in 1.8 ml TE buffer (10 mM of Tris-hydrochloric acid buffer pH8.0, 1 mM of EDTA), whereby a nucleic acid fragment solution was prepared.

### (2) DNA Fractionation

One hundred μl of the nucleic acid fragment solution prepared in (1) and adjusted to 1 mM and three probe nucleic acids (bio-m-Tyr, bio-m-Trp, Sh-bio-m-Tyr) synthesized in Example 1 were placed in 0.5 ml tubes for PCR, respectively, and set in a thermal cycler for PCR (GeneAmp PCR System, manufactured by Applied Biosystems Inc.) and left for five minutes at 95° C., for two minutes at 85° C., for one minute at 75° C., for one minute at 65° C., for one minute at 55° C., for one minute at 45° C., and thereafter at 4° C., whereby dissociation of the nucleic acid fragment into single strands and hybridization with the probe nucleic acid were carried out concurrently.

Five hundreds μl of magnetic carrier (Megacell-Streptavidin, manufactured by CORTEX BIOSYSTEM) was cleaned with 500 μl of 1XB/W buffer, and then only the magnetic carrier was recovered with a recovering magnet (manufactured by Promega) (this is positioned as cleaning operation). This cleaning operation was repeated for three times, and then the magnetic carrier was suspended in 500 μl of 1XB/W buffer. To this, 110 μl of the hybridized DNA solution mentioned above was added and stirred (10 rpm) for ten minutes at room temperature, whereby the probe nucleic acid bound to the magnetic carrier. Thereafter, the magnetic carrier to which the probe nucleic acid (and single-stranded nucleic acid adhering to the probe nucleic acid) was bound was recovered with a magnet, the cleaning operation was repeated for three times, and the nonadherent nucleic acid fragment was removed. To the magnetic carrier to which the probe nucleic acid (and the single-stranded nucleic acid adhering to the probe nucleic acid) thus recovered, 100 μl of 0.1 N sodium hydroxide was added and gently mixed, and then left still for approximately five minutes. The magnetic carrier to which the probe nucleic acid was bound was recovered with the magnet and discarded, and this approximately 100 μl of supernatant was recovered as the anti-sense fraction.

Approximately 100 μl of 0.1 N hydrogen chloride was added to the anti-sense fraction and neutralized. Thereafter, 400 μl of 100% ethanol and 100 μl of 2.5M sodium chloride were added and incubated at −20° C. overnight. This was followed by centrifugation for 15 minutes at 14,000 rpm, and the anti-sense nucleic acid was recovered, cleaned with 70% ethanol, and then dried under reduced pressure. This was then suspended in 15 μl of TE buffer, and a concentration thereof was determined with an absorption spectrometer. As a result, approximately 8 μg of anti-sense nucleic acid was obtained for the respective probe nucleic acids. The anti-sense nucleic acid obtained with the use of bio-m-Tyr as the probe nucleic acid was named as m-Tyr-antiDNA. The anti-sense nucleic acid obtained with the use of bio-m-Trp as the probe nucleic acid was named as m-Trp-antiDNA. The anti-sense nucleic acid obtained with the use of Sh-bio-m-Tyr as the probe nucleic acid was named as Sh-m-Tyr-DNA.

### Example 3

### Blackening Inhibition Effect in Mouse Melanoma Cell—Adding Anti-Sense Nucleic Acid to Medium to Confirm the Effect

A mouse melanoma cell (B16) (allotted by HS Foundation, lot No. JCRB0202) was amplified in a DMEM medium (Dulbecco Modified Eagle Medium containing 10% bovine fetal serum, manufactured by Invitrogen) and added into a 96-hole plate in such a manner that 104 pieces were added per hole. This was cultured in an CO₂ incubator at 37° C., with the use of the DMEM medium containing the m-Tyr-antiDNA prepared in Example 2(2) and having a final concentration of 100 nM and the m-Trp-antiDNA prepared in Example 2(2) and having the final concentration of 1 μM. As a negative control, the nucleic acid fragment (unfractionated nucleic acid) that was double-stranded DNA prepared in Example 2(1) was added so as to be same in concentration. Further, as a comparison, the m-Tyr-oligo and the m-Trp-oligo of the synthetic single-stranded nucleic acid of Example 1 were added so as to be same in concentration.

The mouse melanoma cells were cultured for seven days in 200 μl of DMEM medium containing the nucleic acids, with the medium being changed everyday. Thereafter, the medium was removed, and adherent cells were lightly cleaned with PBS. Then, a PBS solution containing 50 μl of Triton X-100 at a concentration of 0.1% was added and then incubated for one hour at 4° C. Thereafter, 50 μl of 10 mM L-DOPA solution was added and left for approximately one hour at 37° C.

The supernatant was recovered. To measure the degree of blackening with the use of L-DOPA as a substrate, an absorbance at 492 nm was measured with a plate reader (multi scan plus MKII, manufactured by Japan flow laboratories). Results thereof are shown on Table 1. The numerical values on the table indicate remaining activities on the assumption that an activity in the case in which a TE buffer is added in place of a nucleic acid sample is 100%. The lower the numerical value is, the higher the inhibition ratio is. As apparent therefrom, the blackening due to melanin formation is inhibited in a cell to which the anti-sense nucleic acid of Example 2 of the present invention is added.

TABLE 1

| | m-Tyr- | m-Trp- | m-Tyr- | m-Trp- | unfractionated |
|---|---|---|---|---|---|
| | antiDNA | antiDNA | oligo | oligo | nucleic acid |
| 1 μM | 80 | 83 | 9 | 15 | 111 |
| 100 nM | 0 | 0 | 37 | 6 | — |

Further, the m-Tyr-antiDNA, the Sh-m-Tyr-DNA, the m-Tyr-oligo, and the unfractionated nucleic acid of Example 2(1) were respectively added such that the final concentrations became 50 nM and 100 nM, respectively. Examination was carried out in the same manner. Results of this examination are shown on Table 2. The remaining activities on the assumption that the activity in the case in which TE is added in place of the sample is 100%. The numerical values on the table indicate remaining activities on the assumption that an activity in the case in which a TE buffer is added in place of a nucleic acid sample is 100%.

TABLE 2

| | 50 nM | 100 nM |
|---|---|---|
| m-Tyr-antiDNA | 16 | 34 |
| Sh-m-Tyr-DNA | 80 | 64 |
| m-Tyr-oligo | 35 | 14 |
| unfractionated | 90 | 98 |
| nucleic acid | | |

It is also apparent from the results that the anti-sense nucleic acid separated by specific fractionation produces the effect of inhibition.

### Example 4

### Blackeninig Inhibition Effect in Mouse Melanoma Cell—Introduction into Cells

For introduction into cells, SAINT-MIX (manufactured by Takara Bio Inc.) was used. Two μl of 500 μM solution of the m-Tyr-antiDNA prepared in Example 2 and 100 μl of HBS were mixed. Two μl of 500 μM solution of the Sh-m-Tyr-DNA prepared in Example 2 and 100 μl of HBS were mixed. Two μl of 500 μM solution of the unfractionated nucleic acid of Example 2(1) and 100 μl of HBS were mixed. These respective mixtures were left for approximately five minutes at room temperature and then mixed with a mixture of 20 μl SAINT-MIX and 80 μl HBS. Approximately 800 μl of DMEM medium (manufactured by Invitrogen) was added thereto. This mixture was added to a 6-hole plate to which mouse melanoma cells B16 had been added in such a manner that 1×10⁵ cells were added per well. This was incubated in an CO₂ incubator for approximately three hours at 37° C. Then, 2 ml of DMEM medium was added and incubated in the same manner for 24 hours. Thereafter, the medium was discarded, and the cells were peeled off from the plate by use of a trypsin solution (manufactured by Invitrogen) and centrifuged (430×g, 3 minutes) to recover the cells in a centrifuge tube. Then, the degree of blackness was examined visually. Further, in the same manner as in Example 3, a Triton X-100 solution was added to dissolve the cells in the solution, an L-DOPA solution was added, and then the degree of blackening was examined (Table 3). In this case, the lower the absorbance at the wavelength of 492 nm is, the more the blackening is restrained, which absorbency is a value per 10⁶ cells.

TABLE 3

| | | | unfractionated |
|---|---|---|---|
| | m-Tyr-antiDNA | Sh-m-Tyr-DNA | nucleic acid |
| 1 μM | 0.225 | 0.329 | 0.295 |
| 100 nM | 0.227 | 0.281 | 0.311 |

As a result, it was proved that cells into which the anti-sense nucleic acid of the present invention had been introduced restrained the blackening superiorly.

### Example 5

### Synthesis of Probe Nucleic Acid (for Evaluation of Human Melanocyte)

To prepare an anti-sense nucleic acid of a tyrosinase gene that constitutes a human melanin synthetic pathway, the following probe nucleic acid was synthesized. Sequence number 6 shows 48 bases, from 48 to 1 bases upstream from a translation initiation site, of a sense sequence of a sequence of mRNA (Genbank registration number: NM#000372) of Tyr-1 derived from a human. A probe nucleic acid synthesized with biotin being added to the 5′ terminal of Sequence number 6 was named as bio-h-Tyr.

Further, a synthetic single-stranded nucleic acid that was predicted to exhibit anti-sense action with respect to the human tyrosinase gene was prepared as a control, and named as h-Tyr-oligo (sequence number 7). This was the length of 20 bases, from 67 to 48 bases upstream from the translation initiation site of the human tyrosinase gene.

### Example 6

### Blackening Inhibition Effect in Human Melanocyte

In the same manner as in Example 2, bio-h-Tyr was used as a probe nucleic acid and bound to a carrier, whereby an anti-sense nucleic acid was separated from a salmon milt DNA fragment and named as h-Tyr-antiDNA. The degree of inhibition of blackening in the case in which the anti-sense nucleic acid was given to a human normal melanoma cell (manufactured by Kurabo Industries Ltd.) was examined.

Human normal melanocytes (manufactured by Kurabo Industries Ltd.) amplified in a Medium 254 medium (human melanocyte medium to which HMGS was added, manufactured by Kurabo Industries Ltd.) were added into a 96-hole plate in such a manner that 104 pieces were added per hole. The human normal melanocytes were cultured at 37° C. in Medium 254 media containing the h-Tyr-antiDNA at final concentrations of 100 nM and 1 μM, respectively, in an CO₂ incubator. As a control of the synthetic anti-sense nucleic acid, the foregoing was carried out with the h-Tyr-oligo synthesized in Example 5 and added so as to have the same concentration. As a negative control of the synthetic anti-sense nucleic acid, the foregoing was carried out with the unfractionated nucleic acid of Example 2(1) that was added so as to have the same concentration.

The melanocytes were cultured for seven days in 200 μl of Medium 254 media containing the nucleic acids, with the media being changed everyday. Thereafter, the medium was removed, and adherent cells were lightly cleaned with PBS. A PBS solution containing 50 μl of Triton X-100 at a concentration of 0.1% was added and incubated for one hour at 4° C. Then, 50 μl of 10 mM L-DOPA solution was added and left for approximately one hour at 37° C.

A supernatant was recovered, and the degree of blackening at 492 nm was measured with a plate reader (Multiscan Plus MKII, manufactured by Flow Laboratories Japan) using the L-DOPA as a substrate. Results thereof are shown on Table 4. The numerical values on the table indicate remaining activities on the assumption that an activity in the case in which a TE buffer is added in place of a nucleic acid sample is 100%. The lower the numerical value is, the higher the inhibition ratio is. As apparent therefrom, the blackening due to melanin formation is inhibited in a cell to which the anti-sense nucleic acid of the present invention is added.

TABLE 4

| | 100 nM | 1 μM |
|---|---|---|
| h-Tyr-antiDNA | 79 | 30 |
| h-Tyr-oligo | 70 | 31 |
| unfractionated | 93 | 78 |
| nucleic acid | | |

### Example 7

### Synthesis of Probe Nucleic Acid (for Evaluation of Human Fibroblast)

To prepare an anti-sense nucleic acid of human matrix metaloproteinase 1 (MMP-1) gene, the following probe nucleic acid was synthesized. Sequence number 8 shows 48 bases, from 48 to 1 bases upstream from a translation initiation site of a sense sequence of a sequence of mRNA (Genbank registration number: NM#002421) of MMP-1 derived from a human. A probe nucleic acid synthesized with addition of biotin to the 5′ terminal of Sequence number 8 was named as bio-h-MMP1. Further, a synthetic anti-sense nucleic acid that was predicted to exhibit anti-sense action with respect to a human MMP-1 gene was prepared as a control and named as h-MMP1-oligo (sequence number 9). This was the length of 20 bases, from 20 to 1 bases upstream from the translation initiation site of the human tyrosinase gene.

### Example 8

### Inhibition of Human Matrix Metalloproteinase-1 (MMP-1)

### (1) Confirmation by Western Blotting

In the same manner as in Example 2, bio-h-MMP1 was used as a probe nucleic acid, and an anti-sense nucleic acid was separated from a salmon milt DNA fragment by use of a carrier. This was named as h-MMP1-antiDNA. The degree of inhibition of matrix metalloproteinase 1 (MMP-1) in the case in which the anti-sense nucleic acid was given to human normal fibroblast (manufactured by Kurabo Industries Ltd.) was examined.

Human normal fibroblast (manufactured by Kurabo Industries Ltd.) amplified in Medium 106S (manufactured by Kurabo Industries Ltd.) containing 2% FBS was added into a 24-hole plate in such a manner that 5×10⁴ pieces were added per hole, and was cultured in a CO₂ incubator for 24 hours at 37° C. Thereafter, the medium was replaced by a Medium 106S containing no FBS, and UV irradiation (peak wavelength: 352 nm) was carried out for 12 minutes with a UV irradiation apparatus (manufactured by Clinical Supply) under the condition of 3.7 J/cm². Incubation was carried out for 48 hours at 37° C. in an CO₂ incubator in a Medium 106S medium containing the h-MMP1-antiDNA so as to have the final concentration of 1 μM. As a control, h-MMP1-oligo of a synthetic anti-sense nucleic acid was added so as to have the same concentration (the case in which no sample was added served as a negative control).

A cultured supernatant after cultivation was recovered, and the amount of MMP-1 in this sample was confirmed by western blotting. The amount of protein in the sample was determined on the basis of a bovine serum albumin with the user of a MicroBCA assay kit (manufactured by PIRCE). The amount of protein in the sample that is to be applied was adjusted to be the same. In electrophoresis, the sample of the same solution amount was applied with two sheets of gel, one for western blotting and the other for confirmation of CBB staining. Concretely, an electrophoretic sample buffer was added to 5 μg sample, treated with added heat for five minutes at 95° C., and applied to 10% polyacrylamide gel, and electrophoresis was carried out.

Transcription of the gel for western blotting to a polyvinylidene-fluoride membrane (PVDF membrane, Hybond P: manufactured by Amersham Biosciences) was carried out by referring to “semi-dry blotting experimental method: ATTO”.

The PVDF membrane after the transcription was soaked in a blocking solution (0.3% skimmed milk/PBST(Phosphate Buffered Saline with 0.1% Tween20)), and blocked at room temperature for three hours. Thereafter, the membrane was cleaned well with PBST.

The membrane after the blocking was soaked in a solution of an anti MMP-1 (Collagenase-1) antibody (manufactured by LAB VISION) diluted with 0.3% skimmed milk/PBS (Phosphate Buffered Saline) to 10 μg/ml, left to react overnight at 4° C., and then cleaned well with PBST after the end of the reaction.

Next, the membrane was soaked in a goat anti-rabbit IgG polyclonal antibody-peroxidase marker (manufactured by DAKO) solution diluted thousand-fold with 0.3% skimmed milk/PBS, and left to react for two hours at room temperature. After the end of the reaction, the membrane was cleaned well with PBST.

After the cleaning, the membrane was soaked in a substrate solution (ECL Plus Western Blotting Detection System: manufactured by Amersham Biosciences), a film (Hyperfilm ECL: manufactured by Amersham Biosciences) was exposed to chemiluminescence to visualize signals. Further, regarding the gel for confirmation of CBB staining, protein was stained with CBB (Coomassie Brilliant Blue) protein to confirm the amount of protein applied was approximately the same amount.

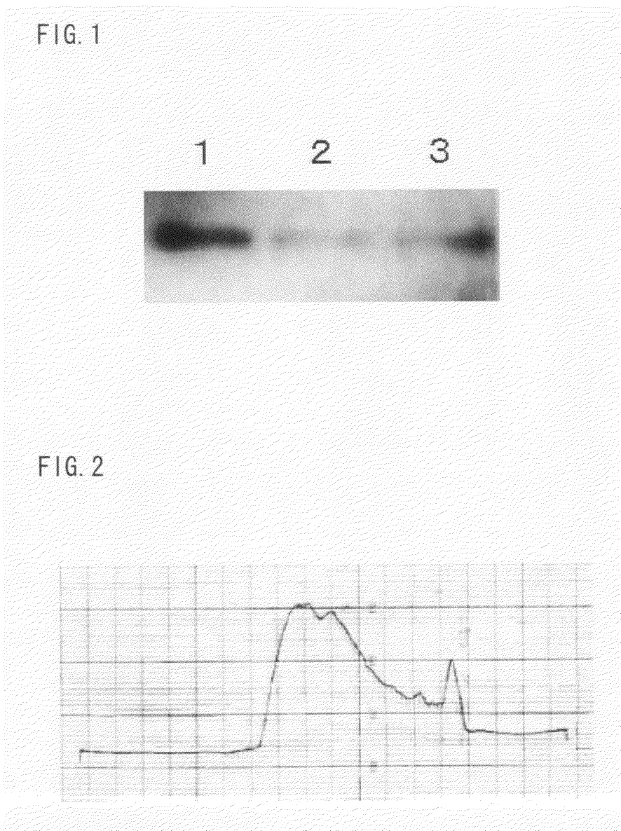

Results of detected bands of MMP-1 of the film thus exposed are shown in FIG. 1. It became apparent from FIG. 1 that protein expression of MMP-1 was inhibited in a cell to which the anti-sense nucleic acid of the present invention was added.

### (2) Confirmation in MMP-1 Activity

The MMP-1 activity was measured with the culture supernatant used in (1), and the degree of inhibition was confirmed. A trypsin having the final concentration of 0.05 mg/mL was added to the supernatant of the medium used in (1) and incubated for 15 minutes at 37° C. Thereafter, a composition for inhibiting a soybean trypsin was added such that the final concentration was brought to 0.25 mg/mL. Then, the type I matrix metaloprotease activity in the supernatant of the medium was measured by using, as a substrate, type I collagen labeled with fluorescein isothiocyanate (FITC). Specifically, 50 μL of 100 mM Tris-hydrochloric acid buffer (pH7.5, 0.4M of sodium chloride, 0.01 M of calcium chloride, and 0.04 (w/v) % sodium azide were contained) containing 0.25 mg/mL FITC labeled I-type collagen was added to 50 μL supernatant, which had been processed with trypsin, of the medium was left to react for two hours at 37° C. with light being blocked. Then, 5 μL of 40 mM o-phenanthroline was added to stop reaction. After the reaction stops, a process of denaturing collagen was carried out for 30 minutes at 37° C. Then, 50 μL of a mixture of ethanol and 0.17 M of Tris-hydrochloric acid buffer (pH9.5, the buffer contains 0.67M of sodium chloride), with the capacitor ratio of 7:3, was added, and only collagen denatured was extracted. This was centrifuged for 15 minutes at 2,000 rpm. A fluorescence intensity of the supernatant was measured at an excitation wavelength of 495 nm and at a fluorescence wavelength of 520 nm. Results thereof are shown on Table 5. Evaluation was carried out on the basis of relative activities with the enzyme activity, which is the negative control, being 100.

TABLE 5

| | relative activity (%) |
|---|---|
| negative control | 100 |
| h-MMP1-antiDNA | 52 |
| h-MMP1-oligo | 41 |

As a result, inhibition by the anti-sense nucleic acid of the present invention was also confirmed in MMP-1 activity.

### Example 9

### Analysis and Fractionation of Anti-Sense Nucleic Acid of the Present Invention

### (1) Preparation of Anti-Sense Nucleic Acid

In the present Example, the m-Tyr-antiDNA and the anti-sense nucleic acid for inhibiting mouse tyrosinase prepared in Example 2(2) were used.

### (2) Chromatographic Fractionation of Anti-Sense Nucleic Acid

Fractionation of the m-Tyr-antiDNA was carried out by use of high-performance liquid chromatography. Specifically, 0.1 ml of m-Tyr-antiDNA solution (concentration 1 mM) was injected into HPLC, and fractionation using an anion exchange column (COSMOSIL DEAE, manufactured by Nacalai) was carried out. Following this injection, cleaning with a buffer (20 mM of Tris-hydrochloric acid buffer, pH8.2) was carried out. Then, elution with the use of a sodium chloride concentration gradient (0 to 1M) was carried out to gather the eluted fractions into the following groups. A chromatographic pattern at this time is shown in FIG. 2 (the horizontal axis is a time period passed, and the vertical axis is an absorption at 254 nm).

As apparent from FIG. 2, it was confirmed that the anti-sense nucleic acid of the present invention exhibited an elution pattern ranging in width and was an aggregate of nucleic acids of various types and sequential sizes.

### (3) Blackening Inhibition Effect in Mouse Melanoma Cell—Confirmed by Adding it to Medium

In the chromatographic fractionation in (2), fractions without a gradient and has a sodium chloride concentration of 0 were fractionated as a control. Fractions having the sodium chloride gradient greater than 0 but not greater than 100 mM were fractionated into (a). Fractions greater than 100 mM but not greater than 200 mM were fractionated into (b). Fractions greater than 200 mM but not greater than 300 mM were fractionated into (c). Fractions greater than 300 mM but not greater than 400 mM were fractionated into (d). Fractions greater than 400 mM but not greater than 500 mM were fractionated into (e). Fractions greater than 500 mM but not greater than 1 M were fractionated into (f). The respective fractions were concentrated with an ethanol precipitate and suspended in a TE buffer.

A mouse melanoma cell (B16) (allotted by HS Foundation, lot No. JCRB0202) was amplified in a DMEM medium (Dulbecco Modified Eagle Medium containing 10% bovine fetal serum, manufactured by Invitrogen) and added into a 96-hole plate in such a manner that 10⁴ pieces were added per hole. The DMEM medium that contained respective suspension liquids of fractions (a) to (f) such that respective final concentrations were brought to 100 nM was added and cultured at 37° C. in an CO₂ incubator. As a control, the m-Tyr-antiDNA was added so as to have the same concentration.

The mouse melanoma cells were cultured for seven days in 200 μl of DMEM media containing the nucleic acids, with the media being changed everyday. Thereafter, the medium was removed, and adherent cells were lightly cleaned with PBS. A PBS solution containing 50 μl of Triton X-100 at a concentration of 0.1% was added and incubated for one hour at 4° C. Then, 50 μl of 10 mM L-DOPA solution was added and left for approximately one hour at 37° C.

A supernatant was recovered, and an absorbance at 492 nm was measured with a plate reader (Multiscan Plus MKII, manufactured by Flow Laboratories Japan), in order to measure the degree of blackening by using L-DOPA as a substrate. Results thereof are shown on Table 6. The numerical values on the table indicate remaining activities on the assumption that an activity in the case in which a TE buffer is added in place of a nucleic acid sample is 100% (this serves as a negative control). The lower the numerical value is, the higher the inhibition ratio is. As apparent therefrom, the blackening due to melanin formation is inhibited in any cells to which the anti-sense nucleic acids of any of fractions (a) to (f) are added.

TABLE 6

| | relative activity (%) |
|---|---|
| negative control | 100 |
| m-Tyr-antiDNA | 53 |
| (a) | 73 |
| (b) | 38 |
| (c) | 48 |
| (d) | 66 |
| (e) | 69 |
| (f) | 76 |

### Example 10

### Synthesis of Long-Strand Probe Nucleic Acid (for Evaluation of Mouse Melanoma)

To prepare an mouse anti-sense nucleic acid targeting a tyrosinase gene constituting a melanin synthetic pathway, the following long-strand probe nucleic acid was synthesized by PCR. Nucleic acids of sequence numbers 10, 11, 12, and 13 are those designed so as to allow the sequence (Genbank registration number: NM#011661), from a transcription initiation site to the 70th base downstream, of a tyrosinase derived from mouse to be synthesized by PCR. They were named as PCR-m-Tyr-1, PCR-m-Tyr-2, PCR-m-Tyr-3, and PCR-m-Tyr-4, respectively. With the use of these nucleic acids, double-stranded DNA from the transcription initiation site to 70 bases downstream was synthesized by PCR to be used as templates for synthesizing the single-stranded long-strand probe nucleic acid by PCR.

Next, with the nucleic acid used as templates, linear PCR was carried out using the synthetic nucleic acid of Sequence number 10 as a primer to synthesize a single-stranded long-strand probe nucleic acid. PCR was carried out under the conditions in which, after denaturalization for two minutes at 94° C., the following reaction cycle was carried out for 100 times: for 30 seconds at 94° C.; for 30 seconds at 55° C.; and for 15 seconds at 72° C. Thereafter, the fragments amplified were recovered with the use of an ethanol precipitate and suspended in 500 μl of sterile water. This long-strand probe nucleic acid was named as L-PCR-m-Tyr (the molecular weight of approximately 21,000).

### Example 11

### Obtaining Anti-Sense Nucleic Acid

(1) Cutting and Fractionating DNA Derived from a Salmon Milt

Twenty ml of 10% (w/v) DNA-Na derived from a salmon milt (manufactured by Nichiro) solution was processed in an ultrasonic wave homogenizer (manufactured by SMT) for 20 minutes to fragment DNA. A solution thereof was injected into an ultrafilter dialysis membrane (manufactured by Nacalai) having the exclusion molecular weight of 2,000, and left overnight in sterile water at room temperature. Thereafter, the content (nucleic acid having the molecular weight of 2,000 or greater) was added to an ultrafilter dialysis membrane (manufactured by Nacalai) having the exclusion molecular weight of 15,000, and left overnight in 40 ml of sterile water at room temperature while being shaken. The DNA fragments (the molecular weight 2,000 to 15,000) contained in the sterile water, which is an external solution, were recovered with an ethanol precipitate and suspended in 4 ml of sterile water. This is the fractionated nucleic acid fragment solution.

### (2) Separating Object Anti-Sense Nucleic Acid

Nine hundreds μl fractionated nucleic acid fragment solution prepared in (1) and adjusted so as to have the concentration of 5 mM and 100 μl of L-PCR-m-Tyr of Example 10 were injected into an Eppendorf tube, placed in a heat block (manufactured by Taitec), and heated for five minutes at 100° C., whereby the fractionated nucleic acid fragments were broken into single strands. This was then maintained for five minutes at 30° C., whereby the long-strand probe nucleic acid and the fractionated nucleic acid fragment were hybridized.

This was added to an ultrafilter membrane (manufactured by Nacalai) having the exclusion molecular weight of 15,000 and left overnight in 4 L of sterile water. Then, the fractionated nucleic acid fragments that not annealed with the long-strand probe nucleic acid and had the molecular weight of 15,000 or below were removed. Thereafter, an ultrafilter membrane containing the probe nucleic acid solution to which the object anti-sense nucleic acid adhered was placed in a container having 5 ml of sterile water, and heated for 10 minutes at 105° C. Then, the single-stranded nucleic acid annealed with the long-strand probe nucleic acid was peeled off and eluted into an external solution to recover the anti-sense nucleic acid. The anti-sense nucleic acid is f-m-Tyr-antiDNA. This recovery was carried out by processing an approximately 5 ml of external solution with the use of an ethanol precipitate method and suspending the precipitate thus obtained in 300 μl of TE buffer.

### (3) Blackening Inhibition Effect in Mouse Melanoma Cells

In the same manner as in Example 3, effect of the anti-sense nucleic acid recovered in (2) was examined on the basis of blackening inhibition with respect to mouse melanoma cells (B16). The anti-sense nucleic acid added were f-m-Tyr-antiDNA of (2) and m-Tyr-antiDNA prepared in Example 2(2), m-Tyr-oligo of the synthetic anti-sense nucleic acid was used as the control, and respective final additive concentrations at the time of application to a cell culture medium were all arranged so as to be brought to 1 μM. Results thereof are shown on Table 7. The numerical values on the table indicate remaining activities on the assumption that an activity in the case in which a TE buffer is added in place of a nucleic acid sample is 100%. The lower the numerical value is, the higher the inhibition ratio is. As apparent therefrom, inhibitory activities that are approximately the same level as those in the method using the magnetic carriers were exhibited in cells to which the anti-sense DNA prepared with the use of the ultrafilter membranes were added.

TABLE 7

| | 1 μM |
|---|---|
| f-m-Tyr-antiDNA | 44 |
| m-Tyr-antiDNA | 34 |
| m-Tyr-oligo | 27 |

The following describes Examples in regard to skin preparations for external use, which preparations adopt an anti-sense nucleic acid of the present invention. In the following Examples, the anti-sense nucleic acid obtained in Example 6 was used. The blending quantities in the following Examples are all in parts by weight.

### Example 12

### Skin Lotion

| | | |
|---|---|---|
| (1) | ethanol | 10.0 |
| (2) | hydroxyethyl cellulose | 1.0 |
| (3) | anti-sense nucleic acid | 0.01 |
| (4) | methyl paraoxybenzoate | 0.1 |
| (5) | purified water | 88.8 |

Preparation method: (1) to (4) were added to (5) one after another and dissolved evenly.

### Example 13

### Skin Emulsion

| | | |
|---|---|---|
| (1) | stearic acid | 0.2 |
| (2) | cetanol | 1.5 |
| (3) | vaseline | 3.0 |
| (4) | liquid paraffin | 7.0 |
| (5) | polyoxyethylene (10E.O.) monooleate ester | 1.5 |
| (6) | tocopherol acetate | 1.0 |
| (7) | glycerin | 5.0 |
| (8) | methyl paraoxybenzoate | 0.1 |
| (9) | triethanolamine | 1.0 |
| (10) | purified water | 79.5 |
| (11) | anti-sense nucleic acid | 0.01 |

Preparation method: Oil-phase components of (1) to (6) were mixed and heated to dissolve evenly, and kept at 70° C. On the other hand, water-phase components of (7) to (10) were mixed and heated to become even and be brought to 70° C. While the water-phase components were stirred, the oil-phase components were gradually added to the water-phase components to be emulsified, and cooled. After this cooling, (11) was added thereto at 40° C. and mixed.

### Example 14

### Skin Gel

| | | |
|---|---|---|
| (1) | dipropylene glycol | 10.0 |
| (2) | carboxy vinyl polymer | 0.5 |
| (3) | potassium hydrate | 0.1 |
| (4) | methyl paraoxybenzoate | 0.1 |
| (5) | purified water | 88.8 |
| (6) | anti-sense nucleic acid | 0.01 |

Preparation method: (2) was evenly dissolved into (5), and then (4) was dissolved into (1) and added. Thereafter, (3) was added to increase viscosity, and (6) was added and mixed.

### Example 15

### Skin Cream

| | | |
|---|---|---|
| (1) | yellow beeswax | 6.0 |
| (2) | cetanol | 5.0 |
| (3) | reduced lanolin | 8.0 |
| (4) | squalan | 27.5 |
| (5) | glyceryl fatty acid ester | 4.0 |
| (6) | oleophilic glycerylmonostearate ester | 2.0 |
| (7) | polyoxyethylene (20E.O.) sorbitanmonolaurate ester | 5.0 |
| (8) | propylene glycol | 5.0 |
| (9) | methyl paraoxybenzoate | 0.1 |
| (10) | purified water | 36.4 |
| (11) | anti-sense nucleic acid | 0.01 |

Preparation method: Oil-phase components of (1) to (7) were mixed, dissolved, and heated to 75° C. On the other hand, water-phase components of (8) to (10) were mixed, dissolved, and heated to 75° C. Then, the oil-phase components were added to the water-phase components, preliminarily emulsified, and then evenly emulsified in a homomixer and cooled. After this cooling, (11) was added at 40° C. and mixed.

### Example 16

### Oil-in-Water Type Emulsifying Ointment

| | | |
|---|---|---|
| (1) | white vaseline | 25.0 |
| (2) | stearyl alcohol | 25.0 |
| (3) | glycerin | 12.0 |
| (4) | sodium lauryl sulfate | 1.0 |
| (5) | methyl paraoxybenzoate | 0.1 |
| (6) | purified water | 36.3 |
| (7) | anti-sense nucleic acid | 0.01 |

Preparation method: Oil-phase components of (1) to (4) were mixed, dissolved evenly, and heated to 75° C. On the other hand, (5) was dissolved into (6) and heated to 75° C., and the oil-phase components were added thereto and emulsified and cooled. After this cooling, (7) was added at 40° C. and mixed.

### Example 17

### Toner

| | | |
|---|---|---|
| (1) | ethanol | 10.00 |
| (2) | 1,3-butylene glycol | 5.00 |
| (3) | anti-sense nucleic acid | 0.01 |
| (4) | perfume | 0.10 |
| (5) | purified water | 84.89 |

Preparation method: (1) to (4) were added to (5) one after another and mixed evenly to be dissolved.

### Example 18

### Essence

| | | |
|---|---|---|
| (1) | glycerin | 2.00 |
| (2) | 1,3-butylene glycol | 3.00 |
| (3) | polyoxyethylene (25E.O.) oleylether | 0.50 |
| (4) | anti-sense nucleic acid | 0.01 |
| (5) | ethanol | 15.00 |
| (6) | methyl paraoxybenzoate | 0.10 |
| (7) | perfume | 0.10 |
| (8) | purified water | 79.25 |

Preparation method: (6) and (7) were dissolved into (5). This was added, together with (1) to (4), to (8) and mixed evenly to be dissolved.

### Example 19

### Emollient Cream (Water-in-Oil Type)

| | | |
|---|---|---|
| (1) | liquid paraffin | 30.00 |
| (2) | microcrystalline wax | 2.00 |
| (3) | vaseline | 5.00 |
| (4) | ester diglyceryldioleate | 5.00 |
| (5) | L-sodium glutamate | 1.60 |
| (6) | L-serine | 0.40 |
| (7) | propylene glycol | 3.00 |
| (8) | methyl paraoxybenzoate | 0.10 |
| (9) | purified water | 52.75 |
| (10) | perfume | 0.10 |
| (11) | anti-sense nucleic acid | 0.01 |

Preparation method: (5) and (6) were dissolved into a part of (9) and adjusted to 50° C., and, while being stirred, gradually added to (4) that had been hated to 50° C. This was evenly dispersed into (1) to (3) that had been mixed, heated to 70° C., and dissolved in advance. While being stirred, (7) and (8) that had been resolved into the remaining portion of (9) and heated to 70° C. were added thereto and emulsified in a homomixer. After cooling, (10) and (11) were added at 40° C. and mixed.

### Example 20

### Makeup Base Cream

| | | |
|---|---|---|
| (1) | stearic acid | 12.00 |
| (2) | cetanol | 2.00 |
| (3) | glyceryltri 2-ethylhexanoate ester | 2.50 |
| (4) | self-emulsified-type glycerylmonostearate ester | 2.00 |
| (5) | propylene glycol | 10.00 |
| (6) | potassium hydrate | 0.30 |
| (7) | purified water | 69.58 |
| (8) | titanium oxide | 1.00 |
| (9) | colcothar | 0.10 |
| (10) | yellow iron oxide | 0.40 |
| (11) | perfume | 0.10 |
| (12) | anti-sense nucleic acid | 0.01 |

Preparation method: Oil-phase components of (1) to (4) were mixed and heated to 75° C. so as to become even. On the other hand, water-phase components of (5) to (7) were mixed, heated to 75° C., and dissolved so as to become even. Pigments of (8) to (10) were added thereto and evenly dispersed in a homomixer. The oil-phase components were added to the water-phase components, emulsified in the homomixer, and then cooled. Thereafter, (11) and (12) were added at 40° C. and mixed.

### Example 21

### Milky Emulsion Foundation

| | | |
|---|---|---|
| (1) | stearic acid | 2.00 |
| (2) | squalan | 5.00 |
| (3) | octyldodecyl myristate | 5.00 |
| (4) | cetanol | 1.00 |
| (5) | decaglycerylmonoisopalmitate ester | 9.00 |
| (6) | 1,3-butylene glycol | 6.00 |
| (7) | potassium hydrate | 0.10 |
| (8) | methyl paraoxybenzoate | 0.10 |
| (9) | purified water | 53.40 |
| (10) | titanium oxide | 9.00 |
| (11) | talc | 7.40 |
| (12) | colcothar | 0.50 |
| (13) | yellow iron oxide | 1.10 |
| (14) | black iron oxide | 0.10 |
| (15) | perfume | 0.15 |
| (16) | anti-sense nucleic acid | 0.01 |

Preparation method: Oil-phase components of (1) to (5) were mixed and heated to 75° C. so as to become even. On the other hand, water-phase components of (6) to (9) were mixed, heated to 75° C., and dissolved so as to become even. Pigments of (10) to (14) were added thereto and evenly dispersed in a homomixer. The oil-phase components were added to the water-phase components, evenly emulsified in a homomixer, and then cooled. (15) and (16) were added at 40° C. and mixed.

### Example 22

### Hand Cream

| | | |
|---|---|---|
| (1) | cetanol | 4.0 |
| (2) | vaseline | 2.0 |
| (3) | liquid paraffin | 10.0 |
| (4) | glyceryl monostearate ester | 1.5 |
| (5) | polyoxyethylene (60E.O.) glyceryl isostearate ester | 2.5 |
| (6) | tocopheryl acetate | 0.5 |
| (7) | glycerin | 20.0 |
| (8) | methyl paraoxybenzoate | 0.1 |
| (9) | purified water | 59.2 |
| (10) | anti-sense nucleic acid | 0.01 |

Preparation method: Oil-phase components of (1) to (6) were mixed, dissolved, and heated to 75° C. On the other hand, water-phase components of (7) to (9) were mixed, dissolved, and heated to 75° C. Thereafter, the oil-phase components were added to the water-phase components, preliminarily emulsified, and then evenly emulsified in a homomixer and cooled. Then, (10) was added at 40° C. and mixed.

1. A method of producing an anti-sense nucleic acid, the method comprising processing a material of a nucleic acid derived from an organism.

2. The method of claim 1, wherein the organism is a microorganism, an animal, or a plant.

3. The method of claim 1, wherein the nucleic acid fragmented and broken into a single strand is made to adhere to the probe nucleic acid having at least a part of a base sequence of a target gene, and, after the nucleic acid adhering to the probe nucleic acid is separated from a nonadherent nucleic acid, the nucleic acid that is single-stranded and adheres to the probe nucleic acid is peeled off from the probe nucleic acid and recovered.

4. The method of claim 3, wherein the probe nucleic acid is fixed to a carrier at least in the step of separating the nucleic acid adhering to the probe nucleic acid.

5. The method of claim 3, wherein an ultrafilter membrane is used in the steps of separating and/or recovering the adherent nucleic acid adhering to the probe nucleic acid.

6. The method of claim 5, comprising the following steps (a) to (d) of:
(a) fragmenting and fractionating the nucleic acid derived from an organism;
(b) dissociating a fragment of the nucleic acid thus fragmented and fractionated into a single strand, and making the fragment adhere to the probe nucleic acid;
(c) separating and removing the nonadherent nucleic acid by use of an ultrafilter membrane; and
(d) peeling off, from a probe nucleic acid, the fragment of the nucleic acid thus dissociated into the single strand, and recovering the fragment by use of the ultrafilter membrane.

7. The method of claim 6, wherein steps (b) to (d) are repeated by use of a probe nucleic acid having a molecular weight greater than an exclusion molecular weight of the ultrafilter membrane used in the steps of separating and recovering.

8. An anti-sense nucleic acid, characterized by being derived from an organism.

9. The anti-sense nucleic acid of claim 8, wherein the organism is a microorganism, an animal, or a plant.

10. An anti-sense nucleic acid, having a range in nucleic acid type.

11. An anti-sense nucleic acid, obtained by use of the method defined in claim 3.

12. The anti-sense nucleic acid defined in claim 8, wherein the anti-sense nucleic acid is to be hybridized with a melanin synthesis pathway related gene, a wrinkle formation related gene, an age-related gene, a hair-growth related gene, or a cancer-related gene.

13. The anti-sense nucleic acid of claim 12, wherein the nucleic acid is to be hybridized with a human tyrosinase gene or a human MMP-1 gene.

14. A cosmetic composition or a skin preparation for external use, each containing, as an effective ingredient, an anti-sense nucleic acid defined in claim 8.

15. A pharmaceutical composition, containing, as an active ingredient, an anti-sense nucleic acid defined in claim 8.


SEQUENCE LISTING (13 sequences)

SEQ ID NO: 1 - DNA, 48 bp, Artificial Sequence
  SYNTHETIC DNA SEQUENCE CORRESPONDING TO 51-4 BASES UPSTREAM OF TRANSLATION INITIATION SITE OF MOUSE TYROSINASE mRNA
tgtgacactcattaacctattggtgcagattttgtatgatctaaagga

SEQ ID NO: 2 - DNA, 48 bp, Artificial Sequence
  SYNTHETIC DNA SEQUENCE CORRESPONDING TO 50-3 BASES UPSTREAM OF TRANSLATION INITIATION SITE OF MOUSE Trp-1 mRNA
gcaaagagcagcataggagacctgtgttctgaactcttgcttcgagaa

SEQ ID NO: 3 - DNA, 48 bp, Artificial Sequence
  SYNTHETIC SEQUENCE THAT IS RANDOMLY-REARRANGED SEQUENCE NUMBER 1
gcgcacttatgggttgttagtttatgaaacaatgatctaagcatattc

SEQ ID NO: 4 - DNA, 20 bp, Artificial Sequence
  CHEMICALLY-SYNTHESIZED ANTI-SENSE NUCLEIC ACID m-Tyr-oligo
accaataggttaatgagtgt

SEQ ID NO: 5 - DNA, 20 bp, Artificial Sequence
  CHEMICALLY-SYNTHESIZED ANTI-SENSE NUCLEIC ACID m-Trp-oligo
tcagaacacaggtctcctat

SEQ ID NO: 6 - DNA, 48 bp, Artificial Sequence
  SYNTHETIC DNA SEQUENCE CORRESPONDING TO 48-1 BASES UPSTREAM OF TRANSLATION INITIATION SITE OF HUMAN Tyr-1 mRNA
gactccaattagccagttcctgcagaccttgtgaggactagaggaaga

SEQ ID NO: 7 - DNA, 20 bp, Artificial Sequence
  CHEMICALLY-SYNTHESIZED ANTI-SENSE NUCLEIC ACID h-Tyr-oligo
cacagatttctctttccagc

SEQ ID NO: 8 - DNA, 48 bp, Artificial Sequence
  SYNTHETIC DNA SEQUENCE CORRESPONDING TO 48-1 BASES UPSTREAM OF TRANSLATION INITIATION SITE OF HUMAN MMP-1 mRNA
tgggaagccatcacttaccttgcactgacaaagaagacaaaggccagt

SEQ ID NO: 9 - DNA, 20 bp, Artificial Sequence
  CHEMICALLY-SYNTHESIZED ANTI-SENSE NUCLEIC ACID h-MMP1-oligo
actggcctttgtcttctttc

SEQ ID NO: 10 - DNA, 20 bp, Artificial Sequence
  SYNTHETIC PCR PRIMER FOR MOUSE TYROSINASE GENE
caaagaagactgtgacactc

SEQ ID NO: 11 - DNA, 35 bp, Artificial Sequence
  SYNTHETIC PCR PRIMER FOR MOUSE TYROSINASE GENE
acaaaatctgcaccaataggttaatgagtgtcaca

SEQ ID NO: 12 - DNA, 35 bp, Artificial Sequence
  SYNTHETIC PCR PRIMER FOR MOUSE TYROSINASE GENE
cctattggtgcagattttgtatgatctaaaggaga

SEQ ID NO: 13 - DNA, 20 bp, Artificial Sequence
  SYNTHETIC PCR PRIMER FOR MOUSE TYROSINASE GENE
caagaacatttctcctttag